SEQUENCE LISTING (61 sequences)

SEQ ID NO: 1 - protein, 1053 aa, Homo sapiens
GYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPTGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRA

SEQ ID NO: 2 - protein, 1038 aa, Homo sapiens
DGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRA

SEQ ID NO: 3 - protein, 378 aa, Sprague Dawley
MFAMDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 4 - DNA, 3359 bp, Homo sapiens
aagtcgaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccacgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagt

SEQ ID NO: 5 - DNA, 3096 bp, Homo sapiens
gatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgat

SEQ ID NO: 6 - DNA, 4416 bp, Artificial Sequence
  Chimeric collagen-encoding sequence
atgttcagctttgtggacctccggctcctgctcctcttaggggccactgccctcctgacgcatggccaagaagacatccctgaagtcagctgcatacacaatggcctaagggtccctaatggtgagacgtggaaacctgatgtatgcttgatctgtatctgccacaatggcacggctgtgtgcgatggcgtgctatgcaaagaagacttggactgtcccaacccccaaaaacgggagggcgagtgctgtcctttctgcccagaagaatatgtatcaccagacgcagaagtcataggagtcgagggacccaagggagaccctggcccccaaggcccacggggacctgttggcccccctggacaagatggcatccctggacagcctggacttcctggtcctcctggtccccccggcccccccggaccccctggtcttggaggaaactttgcttcccagctgtcctatggctatgacgaaaagtcgaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccacgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtctactgcaacatggagacaggtcagacctgtgtgttccccactcagccctctgtgcctcagaagaactggtacatcagcccaaaccccaaggagaagaagcatgtctggtttggagagagcatgaccgatggattccagttcgagtatggaagcgaaggttccgatcctgccgatgtcgctatccagctgaccttcctgcgcctgatgtccaccgaggcctcccagaacatcacctatcactgcaagaacagcgtagcctacatggaccaacagactggcaacctcaagaagtccctgctcctccagggctccaacgagatcgagctcaggggcgaaggcaacagtcgattcacctacagcacgcttgtggatggctgcacgagtcacaccggaacttggggcaagacagtcatcgaatacaaaaccaccaagacctcccgcctgcccatcatcgatgtggctcccttggacattggtgccccagaccaggaattcggaatggacattggccctgcctgcttcgtgcctagggactataaggacgatgatgacaaggactacaaagatgatgacgataaa

SEQ ID NO: 7 - DNA, 4143 bp, Artificial Sequence
  Chimeric collagen-encoding sequence
atgctcagctttgtggatacgcgaactctgttgctgcttgcagtaacgtcgtgcctagcaacatgccaatctttacaaatgggatctgtacggaagggccccactggagacagaggaccgcgtggacaaaggggcccagcaggtccccgaggcagagatggtgttgatggtcccgttggccctcctggtccccctggtgcccctggcccccctggtccccctggcccccctggtcttactgggaatttcgcagcccagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcacagccttcactcagacccaaggactatgaagttgatgcaactctgaaatctctcaataaccaaatcgagacccttctcactcctgaaggctctagaaagaaccctgcccgcacatgccgtgacttaagactcagccacccagagtggaagagcgattactactggattgaccctaaccaaggatgcactatggatgccatcaaagtgtactgcgatttctctactggtgaaacctgcatccaggcccaacctgtcaacaccccagccaagaatgcatacagccgtgcccaggccaacaagcatgtctggttaggagagaccatcaatggtggcagccagtttgaatacaacgcagaaggggtgtcctccaaggaaatggcaactcagctcgccttcatgcgcctgctagccaaccgtgcttctcagaacatcacctaccactgcaagaacagcattgcgtacctggacgaggagacaggccgcctgaataaggctgtcattctgcagggctccaacgacgtcgaacttgttgctgagggcaacagcagattcacctacactgtccttgtcgatggctgctccaaaaagacaaatgaatgggacaagacaatcattgaatacaaaacgaataagccatctcgcctgccattccttgacattgcacctctggacatcggtggtactaaccaagaattccgtgtggaggttggccctgtctgtttcaaatacccatacgatgttccagattacgct

SEQ ID NO: 8 - DNA, 24 bp, Artificial Sequence
  forward primer
taggtgatttctcatcatagccat

SEQ ID NO: 9 - DNA, 24 bp, Artificial Sequence
  reverse primer
aaacatggctatgatgagaaatca

SEQ ID NO: 10 - DNA, 24 bp, Artificial Sequence
  forward primer
taggagtttccgtgcctggcccca

SEQ ID NO: 11 - DNA, 24 bp, Artificial Sequence
  reverse primer
aaactggggccaggcacggaaact

SEQ ID NO: 12 - DNA, 21 bp, Artificial Sequence
  forward primer
tagggctcagtattctgacaa

SEQ ID NO: 13 - DNA, 21 bp, Artificial Sequence
  reverse primer
aaacttgtcagaatactgagc

SEQ ID NO: 14 - DNA, 21 bp, Artificial Sequence
  forward primer
taggtttaacttagtattgtg

SEQ ID NO: 15 - DNA, 21 bp, Artificial Sequence
  reverse primer
aaaccacaatactaagttaaa

SEQ ID NO: 16 - DNA, 27 bp, Artificial Sequence
  forward primer
tacagacaagctgtgaatggcatccct

SEQ ID NO: 17 - DNA, 27 bp, Artificial Sequence
  reverse primer
tcctccggtcgacttttcgtcatagcc

SEQ ID NO: 18 - DNA, 27 bp, Artificial Sequence
  forward primer
aagtcgaccggaggaatttccgtgcct

SEQ ID NO: 19 - DNA, 27 bp, Artificial Sequence
  reverse primer
actttgatggcatccaggttgcagcct

SEQ ID NO: 20 - DNA, 27 bp, Artificial Sequence
  forward primer
ggatgccatcaaagtctactgcaacat

SEQ ID NO: 21 - DNA, 27 bp, Artificial Sequence
  reverse primer
cacgaagcaggcagggccaatgtccat

SEQ ID NO: 22 - DNA, 27 bp, Artificial Sequence
  forward sequence
cctgcctgcttcgtgcctagggactat

SEQ ID NO: 23 - DNA, 27 bp, Artificial Sequence
  forward primer
cgactctagaggatctgctcgaatcga

SEQ ID NO: 24 - DNA, 27 bp, Artificial Sequence
  forward primer
gatcctctagagtcgtgcctggcccca

SEQ ID NO: 25 - DNA, 27 bp, Artificial Sequence
  reverse primer
taccttccagggaattcccaccagtgg

SEQ ID NO: 26 - DNA, 30 bp, Artificial Sequence
  forward primer
aattccctggaaggtagctctcctgagtag

SEQ ID NO: 27 - DNA, 35 bp, Artificial Sequence
  reverse primer
ctgtccagggatgccattcacagcttgtctgtaag

SEQ ID NO: 28 - DNA, 33 bp, Artificial Sequence
  forward primer
tacagacaagctgtgagggtagccgcagccttc

SEQ ID NO: 29 - DNA, 25 bp, Artificial Sequence
  reverse primer
tactgggctgcgaaattctggaggg

SEQ ID NO: 30 - DNA, 25 bp, Artificial Sequence
  forward primer
tttcgcagcccagtatgatggaaaa

SEQ ID NO: 31 - DNA, 25 bp, Artificial Sequence
  reverse primer
ctgtgagcgaggctggtcagccctg

SEQ ID NO: 32 - DNA, 25 bp, Artificial Sequence
  forward primer
ccagcctcgctcacagccttcactc

SEQ ID NO: 33 - DNA, 25 bp, Artificial Sequence
  reverse primer
tttgaaacagacagggccaacctcc

SEQ ID NO: 34 - DNA, 25 bp, Artificial Sequence
  forward primer
cctgtctgtttcaaatacccatacg

SEQ ID NO: 35 - DNA, 25 bp, Artificial Sequence
  reverse primer
ccatggagttttaaccatagagccc

SEQ ID NO: 36 - DNA, 25 bp, Artificial Sequence
  forward primer
gttaaaactccatggttagatcaaa

SEQ ID NO: 37 - DNA, 32 bp, Artificial Sequence
  reverse primer
gtcctactcaggagagccttatagtatattac

SEQ ID NO: 38 - DNA, 32 bp, Artificial Sequence
  forward primer
gtaatatactataaggctctcctgagtaggac

SEQ ID NO: 39 - DNA, 33 bp, Artificial Sequence
  reverse primer
gaaggctgcggctaccctcacagcttgtctgta

SEQ ID NO: 40 - DNA, 21 bp, Artificial Sequence
  forward primer
cataacagccccctccatttc

SEQ ID NO: 41 - DNA, 20 bp, Artificial Sequence
  reverse primer
accagtagcaccatcatttc

SEQ ID NO: 42 - DNA, 18 bp, Artificial Sequence
  forward primer
agcagatcctctagagtc

SEQ ID NO: 43 - DNA, 21 bp, Artificial Sequence
  reverse primer
tggctttggtttaggggaacc

SEQ ID NO: 44 - DNA, 18 bp, Artificial Sequence
  forward primer
cagagatggtgttgatgg

SEQ ID NO: 45 - DNA, 18 bp, Artificial Sequence
  reverse primer
aggtccttggaaaccttg

SEQ ID NO: 46 - DNA, 20 bp, Artificial Sequence
  forward primer
ctgctagccaaccgtgcttc

SEQ ID NO: 47 - DNA, 19 bp, Artificial Sequence
  reverse primer
gagtaatgcccggcattag

SEQ ID NO: 48 - DNA, 520 bp, Artificial Sequence
  Chimeric collagen-encoding sequence
aactttgcttcccagctgtcctatggctatgacgaaaagtcgaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctg

SEQ ID NO: 49 - DNA, 323 bp, Artificial Sequence
  Chimeric collagen-encoding sequence
gagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtctactgcaacatggagacaggtcagacctgtgtgttccccactcagccctctgtgcctcagaagaactggtacatcagcccaaaccccaaggagaagaagcatgtctggtttggagagagcatgaccgatggattccagttcgagtatggaagcgaaggttccgatcctg

SEQ ID NO: 50 - DNA, 447 bp, Artificial Sequence
  Chimeric collagen-encoding sequence
aatttcgcagcccagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgt

SEQ ID NO: 51 - DNA, 521 bp, Artificial Sequence
  Chimeric collagen-encoding sequence
tggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcacagccttcactcagacccaaggactatgaagttgatgcaactctgaaatctctcaataaccaaatcgagacccttctcactcctgaaggctctagaaagaaccctgcccgcacatgccgtgacttaagactcagccacccagagtggaagagcgattactactggattgaccctaaccaaggatgcactatggatgccatcaaagtgtactgcgatttctctactggtgaaacctgcatccagg

SEQ ID NO: 52 - DNA, 20 bp, Artificial Sequence
  forward primer
aactttgcttcccagctgtc

SEQ ID NO: 53 - DNA, 20 bp, Artificial Sequence
  reverse primer
cagcaccagtagcaccatca

SEQ ID NO: 54 - DNA, 20 bp, Artificial Sequence
  forward primer
gagccagcagatcgagaaca

SEQ ID NO: 55 - DNA, 20 bp, Artificial Sequence
  reverse primer
caggatcggaaccttcgctt

SEQ ID NO: 56 - DNA, 20 bp, Artificial Sequence
  forward primer
aatttcgcagcccagtatga

SEQ ID NO: 57 - DNA, 20 bp, Artificial Sequence
  reverse primer
acgtcctctctcaccaggaa

SEQ ID NO: 58 - DNA, 20 bp, Artificial Sequence
  forward primer
tggtatcgctggtcaccatg

SEQ ID NO: 59 - DNA, 20 bp, Artificial Sequence
  reverse primer
cctggatgcaggtttcacca

SEQ ID NO: 60 - protein, 262 aa, Artificial Sequence
  Chimeric protein peptide chain
ANVVRDRDLEVDTTLKSLSQQIENIRSPEGSRKNPARTCRDLKMCHSDWKSGEYWIDPNQGCNLDAIKVYCNMETGQTCVFPTQPSVPQKNWYISPNPKEKKHVWFGESMTDGFQFEYGSEGSDPADVAIQLTFLRLMSTEASQNITYHCKNSVAYMDQQTGNLKKSLLLQGSNEIELRGEGNSRFTYSTLVDGCTSHTGTWGKTVIEYKTTKTSRLPIIDVAPLDIGAPDQEFGMDIGPACFVPRDYKDDDDKDYKDDDDK

SEQ ID NO: 61 - protein, 253 aa, Artificial Sequence
  Chimeric protein peptide chain
RSQPSLRPKDYEVDATLKSLNNQIETLLTPEGSRKNPARTCRDLRLSHPEWKSDYYWIDPNQGCTMDAIKVYCDFSTGETCIQAQPVNTPAKNAYSRAQANKHVWLGETINGGSQFEYNAEGVSSKEMATQLAFMRLLANRASQNITYHCKNSIAYLDEETGRLNKAVILQGSNDVELVAEGNSRFTYTVLVDGCSKKTNEWDKTIIEYKTNKPSRLPFLDIAPLDIGGTNQEFRVEVGPVCFKYPYDVPDYA

(19) United States
(12) Patent Application Publication
(10) Pub. No.: US 2021/0032311 A1
(43) Pub. Date: Feb. 4, 2021

(54) NON-HUMAN GENE-EDITED MAMMAL, PROTEIN CRUDE EXTRACT SEPARATED FROM CONNECTIVE TISSUE OF NON-HUMAN GENE-EDITED MAMMAL, METHOD FOR PROTEIN CRUDE EXTRACT AND USES OF PROTEIN CRUDE EXTRACT

(71) Applicants: Yu-Chun Wu, Tainan City (TW); Pi-Hung Liao, Kaohsiung City (TW); Shih-Chieh Tsai, Tainan City (TW)

(72) Inventors: Yu-Chun Wu, Tainan City (TW); Pi-Hung Liao, Kaohsiung City (TW); Shih-Chieh Tsai, Tainan City (TW)

(21) Appl. No.: 16/646,172

(22) PCT Filed: Sep. 25, 2017
(86) PCT No.: PCT/CN2017/103194

§ 371 Date: Mar. 11, 2020

(51) Int. Cl. C07K 14/78; A01K 67/027; C12N 15/85

(52) U.S. Cl. CPC C07K 14/78; A01K 67/0275; C12N 15/8509; C12N 2015/8518; A01K 2267/01; A01K 2227/105; A01K 2217/052

(57) ABSTRACT

A non-human gene-edited mammal, a protein crude extract isolated from a connective tissue of the non-human gene-edited mammal, a method for preparing the protein crude extract, and uses of the protein crude extract are provided. The method includes: microinjecting a deoxyribonucleic acid sequence construct (DNA construct) containing SEQ ID NO: 4 or SEQ ID NO: 5 into a rat embryo, transplanting the rat embryo into a female rat of the same species to develop into a mature rat, and isolating a protein crude extract from a connective tissue of the mature rat. The protein crude extract includes human type I collagen and a non-native chimeric protein peptide chain.

### BACKGROUND

### 1. Technical Field

The present disclosure relates to a protein crude extract isolated from a non-human gene-edited mammal and from a connective tissue of the non-human gene-edited mammal, and a method and a use thereof.

### 2. Description of Associated Art

As a very important protein in animals, collagen is an important structural protein in extracellular space. Collagen is also the main component of connective tissues, and also exists in tendons, ligaments, skin, cornea and other tissues, accounting for about 20% to 30% of the total protein content of animals.

Collagen is composed of procollagen, which is formed from three independent collagen peptide chains intertwined with each other, wherein such triple helix structure is formed and maintained through hydrogen bonds formed between glycine in the peptide chains. Several procollagens are laterally stacked and thus induce aldol condensation reaction between them and form a covalent bond, which becomes a collagen microfiber; several collagen microfibers undergo a similar reaction to produce a covalent bond to form collagen fibers, and collagen fiber is the basic form of collagen for physiological action.

Collagen is divided into five types. The type I collagen is mainly found in skin, tendons, organs and bones; the type II collagen is mainly found in cartilage; the type III collagen is the main component of reticulin; the type IV collagen mainly constitutes basal laminin, which is present in the basement membrane of epithelial tissue; and the type V collagen is present on the cell surface, or in hair or placenta.

Collagen has extremely high economic value and can be widely applied in medical or cosmetic products. For example, it can be used as support materials for stem cell culture system to promote stem cell growth, drug-coated materials on drug-containing cardiovascular stents, dressings for burn wounds, collagen hemostatic cotton tablets, collagen membrane bone filler, moisturizing anti-aging cosmetics, oral nutritional supplements, and so on. Currently, in the market of collagen products, the type I collagen are used in most of the raw materials.

At present, the source of collagen is mainly extracted from animal tissues. Collagen commonly found in the market can be extracted from pig skin, cowhide, fish skin, fish scales, and so on. For example, Taiwan Patent No. 1487711 relates to extraction of collagen based on tuna skin. However, animal-derived collagen may cause allergy. For example, people who are allergic to seafood may not be suitable for collagen products extracted from fish skin/fish scales. In order to reduce collagen-induced allergy, US Patent Application No. 2012/0284817A1 utilizes genetic engineering methods to synthesize human collagen in transgenic plants. However, since the enzymatic systems of plants and mammals are not completely identical, the resulting collagen is still different from the collagen generally derived from mammals in the state of the post-translational modification.

In addition, Chinese Patent Application No. 101812457A discloses the use of E. coli in expression of human collagen fragments. However, the pyrogen produced by bacteria makes the expressed product difficult to be applied in clinical practice. The target protein is usually expressed in the form of inclusion bodies, which make the purification of the product difficult. In addition, the post-translational modification of the prokaryotic expression system is imperfect, such that the biological activity of the expressed product is low. In order to overcome the safety problems of endotoxin and pyrogen in bacterial expression systems, more and more researchers begin to use eukaryotic microorganisms to express recombinant human collagen. For example, Chinese Patent Application No. 102020716A discloses the use of yeast cells in expression of recombinant human collagen. However, the expression of human collagen by eukaryotic cells still has problems of purification, low purity, and protein degradation. In addition, the amino acid sequences of these collagens expressed by eukaryotic cells are not completely identical to those in the human body, and there are also defects in biosafety and biocompatibility.

### SUMMARY

In view of the above-mentioned conventional preparation of collagen by the genetically modified organisms, which still has various defects in practical use, the inventors improve them and obtain the present disclosure.

The present disclosure provides a protein crude extract isolated from a connective tissue of a non-human mammal, wherein the non-human mammal has a genomic DNA comprising a deoxyribonucleic acid sequence encoding a chimeric collagen peptide chain and does not have wild-type collagen of the non-human mammal, and the protein crude extract includes human type I collagen and a non-native chimeric protein peptide chain.

In the above protein crude extract, the protein crude extract further comprises β-actin of the non-human mammal.

In the above protein crude extract, the amino acid sequence of the non-native chimeric protein peptide chain comprises SEQ ID NO: 60 or SEQ ID NO: 61.

In the above protein crude extract, the chimeric collagen peptide chain comprises at least one portion of an amino acid sequence of the human type I collagen, and at least one portion of an amino acid sequence of N- and/or C-terminus of the wild-type collagen of the non-human mammal

In the above protein crude extract, the deoxyribonucleic acid sequence comprises at least one deoxyribonucleic acid sequence selected from the group consisting of SEQ ID NO: 48, SEQ ID NO: 49, SEQ ID NO: 50, SEQ ID NO: 51, SEQ ID NO: 6 and SEQ ID NO: 7.

In the above protein crude extract, the deoxyribonucleic acid sequence comprises the deoxyribonucleic acid sequence of SEQ ID NO: 4 or SEQ ID NO: 5.

In the above protein crude extract, the deoxyribonucleic acid sequence comprises the deoxyribonucleic acid sequence of SEQ ID NO: 4 or SEQ ID NO: 5.

In the above protein crude extract, the protein crude extract comprises: (i) an amino acid sequence having a similarity of at least 91% as compared with SEQ ID NO: 1 and an amino acid sequence of SEQ ID NO: 60; (ii) an amino acid sequence having a similarity of at least 93% as compared with SEQ ID NO: 2 and an amino acid sequence of SEQ ID NO: 61; or (iii) an amino acid sequence having a similarity of at least 91% as compared with SEQ ID NO: 1, an amino acid sequence having a similarity of at least 93% as compared with SEQ ID NO: 2, and an amino acid sequence of SEQ ID NO: 60 or SEQ ID NO: 61.

In the above protein crude extract, the protein crude extract comprises: (i) an amino acid sequence of SEQ ID NO: 1 and an amino acid sequence of SEQ ID NO: 60; (ii) an amino acid of SEQ ID NO: 2 and an amino acid sequence of SEQ ID NO: 61; or (iii) an amino acid sequence of SEQ ID NO: 1, an amino acid sequence of SEQ ID NO: 2, and an amino acid sequence of SEQ ID NO: 60 or SEQ ID NO: 61.

In the above protein crude extract, the protein crude extract is specifically recognized by an anti-human collagen antibody.

The present disclosure also provides a method for preparing a protein crude extract containing a target amino acid sequence, comprising: (a) constructing a DNA sequence construct encoding human type I collagen; (b) microinjecting the DNA sequence construct into a non-human mammalian embryo, and then transplanting the non-human mammalian embryo into a female animal of the same species to develop the non-human mammalian embryo into a mature animal, wherein the mature animal has a genomic DNA comprising a deoxyribonucleic acid sequence encoding a chimeric collagen peptide chain and does not have wild-type collagen of the non-human mammal; and (c) isolating a protein crude extract containing the target amino acid sequence from the connective tissue of the mature animal, wherein the target amino acid sequence comprises human type I collagen.

In the above method, the target amino acid sequence further comprises β-actin of the mature animal.

In the above method, the DNA sequence construct comprises the deoxyribonucleic acid sequence of SEQ ID NO: 4 or SEQ ID NO: 5.

In the above method, the target amino acid sequence comprises: (i) an amino acid sequence having a similarity of at least 91% as compared with SEQ ID NO: 1 and an amino acid sequence of SEQ ID NO: 60; (ii) an amino acid sequence having a similarity of at least 93% as compared with SEQ ID NO: 2 and an amino acid sequence of SEQ ID NO: 61; or (iii) an amino acid sequence having a similarity of at least 91% as compared with SEQ ID NO: 1, an amino acid sequence having a similarity of at least 93% as compared with SEQ ID NO: 2, and an amino acid sequence of SEQ ID NO: 60 or SEQ ID NO: 61.

In the above method, the target amino acid sequence comprises: (i) an amino acid sequence of SEQ ID NO: 1 and an amino acid sequence of SEQ ID NO: 60; (ii) an amino acid sequence of SEQ ID NO: 2 and an amino acid sequence of SEQ ID NO: 61; or (iii) an amino acid sequence of SEQ ID NO: 1, an amino acid sequence of SEQ ID NO: 2, and an amino acid sequence of SEQ ID NO: 60 or SEQ ID NO: 61.

In the above method, the chimeric collagen peptide chain comprises at least one portion of an amino acid sequence of the human type I collagen, and at least one portion of an amino acid sequence of N-terminal and/or C-terminal peptide of the wild-type collagen of the non-human mammal

In the above method, the deoxyribonucleic acid sequence encoding the chimeric collagen peptide chain comprises at least one deoxyribonucleic acid sequence selected from the group consisting of SEQ ID NO: 48, SEQ ID NO: 49, SEQ ID NO: 50, SEQ ID NO: 51, SEQ ID NO: 6 and SEQ ID NO: 7.

In the above method, the non-human mammal is rat.

In the above method, the protein crude extract is specifically recognized by an anti-human collagen antibody.

The present disclosure also provides a use of a protein crude extract having a target amino acid sequence in the preparation of human type I collagen, wherein the protein crude extract is isolated from a connective tissue of a non-human mammal. The genomic DNA of the non-human mammal has a deoxyribonucleic acid sequence encoding a chimeric collagen peptide chain. The non-human mammal does not have wild-type collagen of the non-human mammal, and the target amino acid sequence includes human type I collagen.

In the above use, the target amino acid sequence further comprises β-actin of the non-human mammal.

In the above use, the target amino acid sequence comprises: (i) an amino acid sequence having a similarity of at least 91% as compared with SEQ ID NO: 1 and an amino acid sequence of SEQ ID NO: 60; (ii) an amino acid sequence having a similarity of at least 93% as compared with SEQ ID NO: 2 and an amino acid sequence of SEQ ID NO: 61; or (iii) an amino acid sequence having a similarity of at least 91% as compared with SEQ ID NO: 1, an amino acid sequence having a similarity of at least 93% as compared with SEQ ID NO: 2, and an amino acid sequence of SEQ ID NO: 60 or SEQ ID NO: 61.

In the above use, the target amino acid sequence comprises: (i) an amino acid sequence of SEQ ID NO: 1 and an amino acid sequence of SEQ ID NO: 60; (ii) an amino acid sequence of SEQ ID NO: 2 and an amino acid sequence of SEQ ID NO: 61; or (iii) an amino acid sequence of SEQ ID NO: 1, an amino acid sequence of SEQ ID NO: 2, and an amino acid sequence of SEQ ID NO: 60 or SEQ ID NO: 61.

In the above use, the chimeric collagen peptide chain comprises at least one portion of an amino acid sequence of the human type I collagen, and at least one portion of an amino acid sequence of N-terminal and/or C-terminal peptide of wild-type collagen of the non-human mammal.

In the above use, the deoxyribonucleic acid sequence comprises at least one deoxyribonucleic acid sequence selected from the group consisting of SEQ ID NO: 48, SEQ ID NO: 49, SEQ ID NO: 50, SEQ ID NO: 51, SEQ ID NO: 6, and SEQ ID NO: 7.

In the above use, the deoxyribonucleic acid sequence comprises a deoxyribonucleic acid sequence of SEQ ID NO: 4 or SEQ ID NO: 5.

In the above use, the protein crude extract is specifically recognized by an anti-human collagen antibody.

In the above use, the human type I collagen is used in a medical material, a cosmetic care product, and a food additive.

In the above use, the medicinal material comprises collagen hemostatic cotton, surgical suture, wound dressing, bone filler, artificial blood vessel and soft tissue filler.

In the above use, the cosmetic product is in the form of a liquid, an emulsion, a cream, a powder, a whitening agent, a spotting agent, a freckle agent, or any combination thereof.

In the above use, the cosmetic care product comprises a collagen moisturizing liquid, a collagen moisturizing cream and a collagen facial mask.

In the above use, the food additive is added to a bone joint health food and an anti-aging health food.

The present disclosure also provides a non-human gene-edited mammal having a genomic DNA comprising a deoxyribonucleic acid sequence encoding a chimeric collagen peptide chain, wherein the non-human gene-edited mammal does not have wild-type collagen of the non-human gene-edited mammal.

In the above non-human gene-edited mammal, the chimeric collagen peptide chain comprises at least one portion of an amino acid sequence of human type I collagen, and at least one portion of an amino acid sequence of N-terminal and/or C-terminal peptide of wild-type collagen of the non-human gene-edited mammal.

In the above non-human gene-edited mammal, the deoxyribonucleic acid sequence comprises at least one deoxyribonucleic acid sequence selected from the group consisting of SEQ ID NO: 48, SEQ ID NO: 49, SEQ ID NO: 50, SEQ ID NO: 51, SEQ ID NO: 6 and SEQ ID NO: 7.

In the above non-human gene-edited mammal, the non-human gene-edited mammal comprises: (i) an amino acid sequence having a similarity of at least 91% as compared with SEQ ID NO: 1 and an amino acid sequence of SEQ ID NO: 60; (ii) an amino acid sequence having a similarity of at least 93% as compared with SEQ ID NO: 2 and an amino acid sequence of SEQ ID NO: 61; or (iii) an amino acid sequence having a similarity of at least 91% as compared with SEQ ID NO: 1, an amino acid having a similarity of at least 93% as compared with SEQ ID NO: 2, and an amino acid sequence of SEQ ID NO: 60 or SEQ ID NO: 61.

In the above non-human gene-edited mammal, the non-human gene-edited mammal comprises: (i) an amino acid sequence of SEQ ID NO: 1 and an amino acid sequence of SEQ ID NO: 60; (ii) an amino acid sequence of SEQ ID NO: 2 and an amino acid sequence of SEQ ID NO: 61; or (iii) an amino acid sequence of SEQ ID NO: 1, an amino acid sequence of SEQ ID NO: 2 and an amino acid sequence of SEQ ID NO: 60 or SEQ ID NO: 61.

In the above non-human gene-edited mammal, the non-human gene-edited mammal is a rat, a mouse, a pig, a rabbit, a sheep, a goat, a cat, a dog, a calf or a baboon.

In the above non-human gene-edited mammal, the non-human gene-edited mammal is a rat.

### BRIEF DESCRIPTION OF THE DRAWINGS

FIG. 1A shows a schematic diagram of the chimeric genes of the rat collagen gene COL1A1 and the human collagen gene hCOL1A1.

FIG. 1B shows a schematic diagram of the chimeric genes of the rat collagen gene COL1A2 and the human collagen gene hCOL1A2.

FIG. 2 shows the breeding strategy I for gene-edited rat.

FIG. 3 shows the breeding strategy II for gene-edited rat.

FIG. 4 shows the result of gel electrophoresis of the polymerase chain reaction, in which columns 1, 3, 5 and 7 refer to wild-type rat's DNA used as a template; columns 2, 4, 6 and 8 refer to gene-edited rat's DNA used as a template; columns 1 and 2 refer to the sequence of SEQ ID NO: 48; columns 3 and 4 refer to the sequence of SEQ ID NO: 49; columns 5 and 6 refer to the sequence of SEQ ID NO: 50; and columns 7 and 8 refer to the sequence of SEQ ID NO: 51.

FIG. 5 shows the crude extract from the tails of gene-edited rat and wild-type rat.

FIG. 6 shows the analysis results of the tail crude extract based on (A) non-denaturing polyacrylamide gel electrophoresis, and (B) Western blotting.

FIG. 7 shows the rat β-actin presented in the tail rat crude extract detected by Western blotting.

FIG. 8 shows the non-native chimeric protein peptide chain presented in the tail crude extract detected by Western blotting, wherein columns 1, 2, 9 and 10 refer to the tail crude extracts of the wild-type rat; columns 3 and 4 refer to the tail crude extracts of hCOL1A1 gene-edited rat; columns 7 and 8 refer to the tail crude extracts of hCOL1A2 gene-edited rat; columns 5, 6, 11 and 12 refer to the tail crude extracts of hCOL1A1/hCOL1A2 gene-edited rat.

### DETAILED DESCRIPTION OF THE EMBODIMENTS

The following examples are intended to provide a more complete understanding of the present disclosure for a person having ordinary skill in the art and are not intended to limit the present disclosure in any way.

The present disclosure provides a protein crude extract isolated from a connective tissue of a non-human mammal, wherein a genomic DNA of the non-human mammal has a deoxyribonucleic acid sequence encoding a chimeric collagen peptide chain. The non-human mammal has no wild-type collagen of the non-human mammal, and the protein crude extract includes human type I collagen and a non-native chimeric protein peptide chain. The deoxyribonucleic acid sequence comprises at least one deoxyribonucleic acid sequence selected from the group consisting of SEQ ID NO: 48, SEQ ID NO: 49, SEQ ID NO: 50, SEQ ID NO: 51, SEQ ID NO: 6 and SEQ ID NO: 7, and the protein crude extract comprising: (i) an amino acid sequence having a similarity of at least 91% as compared with SEQ ID NO: 1 and an amino acid sequence of SEQ ID NO: 60; or (ii) an amino acid sequence having a similarity of at least 93% as compared with SEQ ID NO: 2 and an amino acid sequence of SEQ ID NO: 61; or (iii) an amino acid sequence having a similarity of at least 91% as compared with SEQ ID NO: 1, an amino acid sequence having a similarity of at least 93% as compared with SEQ ID NO: 2, and an amino acid sequence of SEQ ID NO: 60 or SEQ ID NO: 61. The protein crude extract can be specifically recognized by an anti-human collagen antibody and isolated from a connective tissue of a gene-edited rat.

The present disclosure also provides a method for preparing a protein crude extract containing a target amino acid sequence, comprising: (a) constructing a DNA sequence construct encoding human type I collagen; (b) microinjecting the DNA sequence construct into a non-human mammalian embryo, and then transplanting the non-human mammalian embryo into a female animal of the same species to develop the non-human mammalian embryo into a mature animal, wherein the genome DNA of the mature animal has a deoxyribonucleic acid sequence encoding a chimeric collagen peptide chain, and the mature animal does not have wild-type collagen of the non-human mammal; (c) isolating the protein crude extract comprising the target amino acid sequence from a connective tissue of the mature animal, wherein the target amino acid sequence is human type I collagen and β-actin of the mature animal. The DNA sequence construct comprises the deoxyribonucleic acid sequence of SEQ ID NO: 4 or SEQ ID NO: 5, and the target amino acid sequence is selected from (i) an amino acid sequence having a similarity of at least 91% as compared with SEQ ID NO: 1 and an amino acid sequence of SEQ ID NO: 60; or (ii) an amino acid sequence having a similarity of at least 93% as compared with SEQ ID NO: 2 and an amino acid sequence of SEQ ID NO: 53; or (iii) an amino acid sequence having a similarity of at least 91% as compared with SEQ ID NO: 1, an amino acid sequence having a similarity of at least 93% as compared with SEQ ID NO: 2, and an amino acid sequence of SEQ ID NO: 61 or SEQ ID NO: 53. The chimeric collagen peptide chain includes at least one portion of an amino acid sequence of the human type I collagen, and at least one portion of an amino acid sequence of N-terminal and/or C-terminal peptide of the wild-type collagen of the non-human mammal. The deoxyribonucleic acid sequence encoding a chimeric collagen peptide chain comprises at least one deoxyribonucleic acid sequence selected from the group consisting of SEQ ID NO: 48, SEQ ID NO: 49, SEQ ID NO: 50, SEQ ID NO: 51, SEQ ID NO: 6 and SEQ ID NO: 7. The non-human mammal is a rat, and the prepared protein crude extract is specifically recognized by an anti-human collagen antibody.

For example, the target amino acid contained in the protein crude extract comprises (i) the amino acid sequence of SEQ ID NO: 1 and the amino acid sequence of SEQ ID NO: 60; or (ii) the amino acid sequence of SEQ ID NO: 2 and the amino acid sequence of SEQ ID NO: 61; or (iii) the amino acid sequence of SEQ ID NO: 1, the amino acid sequence of SEQ ID NO: 2, and the amino acid sequence of SEQ ID NO: 60 or SEQ ID NO: 61.

The present disclosure also provides a use of a protein crude extract having a target amino acid sequence for preparing human type I collagen, wherein the target amino acid sequence is selected from (i) an amino acid sequence having a similarity of at least 91% as compared with SEQ ID NO: 1 and the amino acid sequence of SEQ ID NO: 60; or (ii) an amino acid sequence having a similarity of at least 93% as compared with SEQ ID NO: 2 and an amino acid sequence of SEQ ID NO: 61; or (iii) an amino acid sequence having a similarity of at least 91% as compared with SEQ ID NO: 1, an amino acid sequence having a similarity of at least 93% as compared with SEQ ID NO: 2, and an amino acid sequence of SEQ ID NO: 60 or SEQ ID NO: 61. The protein crude extract is specifically recognized by the anti-human collagen antibody. The protein crude extract is isolated from the connective tissue of the gene-edited rat, and the genomic DNA of the gene-edited rat has a deoxyribonucleic acid encoding human type I collagen sequence of SEQ ID NO: 4 or SEQ ID NO: 5.

The present disclosure also provides a non-human gene-edited mammal, wherein the genomic DNA of the non-human gene-edited mammal has a deoxyribonucleic acid sequence encoding a chimeric collagen peptide chain, and the non-human gene-edited mammal does not have wild-type collagen of the non-human gene-edited mammal. The chimeric collagen peptide chain comprises at least one portion of an amino acid sequence of human type I collagen, and at least one portion of an amino acid sequence of N-terminal and/or C-terminal peptide of wild-type collagen of the non-human gene-edited mammal, and the deoxyribonucleic acid sequence comprises at least one deoxyribonucleic acid sequence selected from the group consisting of SEQ ID NO: 48, SEQ ID NO: 49, SEQ ID NO: 50, SEQ ID NO: 51, SEQ ID NO: 6 and SEQ ID NO: 7. The non-human gene-edited mammal comprises: (i) an amino acid sequence having a similarity of at least 91% as compared with SEQ ID NO: 1 and an amino acid sequence of SEQ ID NO: 60; or (ii) an amino acid sequence having a similarity of at least 93% as compared with SEQ ID NO: 2 and an amino acid sequence of SEQ ID NO: 61; or (iii) an amino acid sequence having a similarity of at least 91% as compared with SEQ ID NO: 1, an amino acid sequence having a similarity of at least 93% as compared with SEQ ID NO: 2, and an amino acid sequence of SEQ ID NO: 60 or SEQ ID NO: 61. The non-human gene-edited mammal is rat, mouse, pig, rabbit, sheep, goat, cat, dog, calf or baboon.

In addition, the scope of the present disclosure can be further clarified by the following embodiments, but is not limited in any form.

Briefly, the present disclosure provides a mixture derived from a gene-edited rat expressing exogenous collagen, containing a crude extract of an exogenous collagen and a crude extract of rat β-actin, and provides a preparation method and use thereof. The exogenous collagen gene can be a collagen gene of human, rabbit, bovine or pig, in which the collagen COL1A1 amino acid sequence of rabbit, bovine or pig has a similarity of at least 91% as compared with the human collagen COL1A1 amino acid sequence; the collagen COL1A2 amino acid sequence of rabbit, bovine or pig has a similarity of at least 93% as compared with the human collagen COL1A2 amino acid sequence. The present disclosure also provides a non-human gene-edited mammal, in which the genomic DNA has a deoxyribonucleic acid sequence encoding a chimeric collagen peptide chain. The deoxyribonucleic acid sequence comprises at least one deoxyribonucleic acid sequence selected from the group consisting of SEQ ID NO: 48, SEQ ID NO: 49, SEQ ID NO: 50, SEQ ID NO: 51, SEQ ID NO: 6, and SEQ ID NO: 7, and the non-human gene-edited mammal does not have wild-type collagen of the non-human gene-edited mammal. The following are examples of human collagen gene-edited rat.

### Experiment 1: Preparation of Human Collagen Gene-Edited Rat

This experiment used CRISPR/Cas9 (Clustered Regularly Interspaced Short Palindromic Repeats/Cas9) or TALEN (transcription activator-like effector nucleases) gene editing method to deliver human type I collagen gene hCOLA1 or hCOLA2 into SD rat (Sprague-Dawley rat), such that the rat's connective tissue expressed human type I collagen. The process was as follows:

(i) Preparation of sgRNA (Single Guide RNA), Cas9 Expression RNA, TALEN, COL1A1 or COL1A2 Vector

An oligonucleotide (oligo DNA) recognizing the rat collagen gene COL1A1 or CAL1A2 was synthesized to prepare sgRNA (single guide RNA), wherein the sequences thereof were SEQ ID NO: 8 to SEQ ID NO: 15. The pair of oligo DNA was annealed under the following conditions: 95° C. for 3 minutes, 95° C. for 30 seconds, cooling from 95° C. to 85° C. (cooling rate: 2° C. per minute), cooling from 85° C. to 25° C. (cooling rate: 0.1° C. per second), and then cooled down to 4° C.

The plasmid pUC57-T7-sgRNA was cleaved with restriction enzyme (BsaI) to obtain a linear double-stranded nucleic acid, and the linear double-stranded nucleic acid was ligated with the oligo DNA pair obtained by the above annealing through the nucleic acid ligase to obtain a recombinant plasmid. This plasmid was transformed into Escherichia coli. Since the recombinant plasmid carried a special antibiotic gene. The strain carrying the recombinant plasmid can be selected by the antibiotic, and the strain can be cultured to amplify the above recombinant plasmid. Further, the recombinant plasmid was cleaved with restriction endonuclease HindIII to obtain a linear recombinant plasmid. In addition, Cas9 protein expression plasmid (Cas9 expressing plasmid) was cleaved with restriction endonucleases (XmaI and MfeI) to obtain a linear Cas9 protein expression plasmid. The linear recombinant plasmid and the linear Cas9 protein expression plasmid were subjected to reverse transcription in vitro, and the obtained sgRNA and Cas9 mRNA were then concentrated and purified for microinjection.

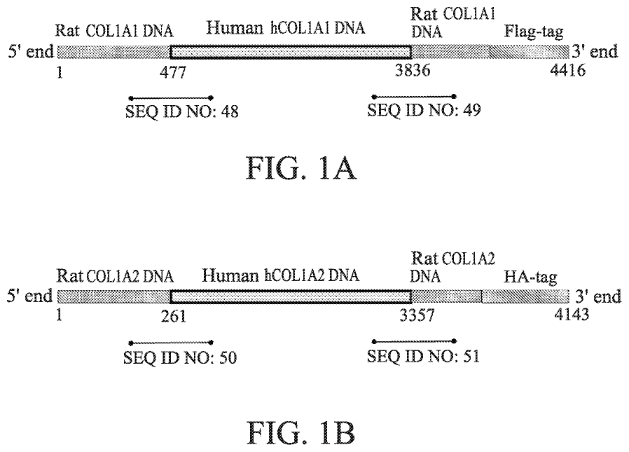

Rat genomic DNA, human cDNA (derived from human oral mucosal cells), rat cDNA, Flag Tag/BGHpA-containing plasmid and ColE1_Amp+Minimal vector were used as templates, and SEQ ID NO: 16 to SEQ ID NO: 27 were used as primers to carry out polymerase chain reaction (PCR) and Gibson Assembly. The obtained recombinant vector ColE1_Amp+Minimal vector_r/hCOL1A1 target construct carried the chimeric gene of rat collagen gene COL1A1 and human collagen gene hCOL1A1. The chimeric gene comprises the 5′ end sequence of the rat collagen gene COL1A1, a part of the human collagen gene hCOL1A1 sequence, the 3′ end sequence of the rat collagen gene COL1A1, and the Flag Tag sequence. After the COL1A1 vector was delivered into the rat, the expressed gene was SEQ ID NO: 6, as shown in FIG. 1A. The COL1A1 vector can also be delivered into the rat by using a pair of commercially synthesized TALEN mRNA. The sites identified by this pair of TALENs were: ATGATGAGAAATCAGCTGG and CTGGTGGCTCACCATGGGG. The 5′ end sequence of the rat collagen gene COL1A1 and the human collagen gene hCOL1A1 sequence have the deoxyribonucleic acid sequence of SEQ ID NO: 48 between them, and the 3′ end sequence of the rat collagen gene COL1A1 and the human collagen gene hCOL1A1 sequence have the deoxyribonucleic acid sequence of SEQ ID NO: 49 between them.

By using primers of SEQ ID NO: 28 to SEQ ID NO: 39, and using rat genomic DNA, human cDNA (derived from human oral mucosal cells), rat cDNA, HA Tag/BGHpA-containing plasmid, and ColE1_Amp+Minimal vector as templates, the polymerase chain reaction and Gibson Assembly were performed. The obtained recombinant vector ColE1_Amp+Minimal vector_r/hCOL1A2 target construct carried a chimeric gene of the rat collagen gene COL1A2 and the human collagen gene COL1A2, and the chimeric gene comprises the 5′ end sequence of rat collagen gene COL1A2, a part of the human collagen gene COL1A2 sequence, the 3′ end sequence of rat collagen gene COL1A2 and the HA tag sequence. After the COL1A2 vector was delivered into the rat, the expressed gene was SEQ ID NO: 7, as shown in FIG. 1B. The COL1A2 vector can also be delivered into the rat by using the two commercially synthesized TALEN mRNA groups. The sites identified by the two TALEN were: Group 1, GTGTGTTTTATTCCCTCCAG and TCCTTTGTCAGAATACTGAG; and Group 2, GAGTTTACATTAATCCTCAC and CATTTGATCTAACCATGGAG. The 5′ end sequence of the rat collagen gene COL1A2 and the human collagen gene COL1A2 sequence have the deoxyribonucleic acid sequence of SEQ ID NO: 50 between them, and the 3′ end sequence of the rat collagen gene COL1A2 and human collagen gene COL1A2 sequence have the deoxyribonucleic acid sequence of SEQ ID NO: 51 between them.

### (ii) Animal Pronuclear Microinjection or Embryonic Nuclear Electroporation (Electroporation)

A zygote on 0.5 day of fertilization was provided. By using pronuclear microinjection or pronuclear electroporation, the product prepared by experiment 1-(i) was delivered into the zygote by 10 ng of sgRNA and 50 ng of Cas9 mRNA, or by mixing the product prepared by 1-(i) with 50 ng of TALEN mRNA and 4 ng of COL1A1 (or COL1A2) vector, so as to obtain the gene-edited zygote on 0.5 day of fertilization.

Sexually mature (full seven weeks) female rat who was estrus and not pregnant was mated with ligation male rat, and such female rat would enter a pseudopregnant state. Next day after the mating, the vagina of the female rat was examined, and the rat who had plug was identified as 0.5 day pseudopregnant female rat. 20-30 gene-edited zygotes were injected into the oviduct of the 0.5 day pseudopregnant female rat, allowing the gene-edited zygote to grow and differentiate in the female rat. The gene-edited rat would be born after 17-21 days, and can be weaned about three weeks after birth. After weaning, the tail of the gene-edited rat was collected to extract DNA for performing genotyping and selecting the successfully gene-edited preliminary generation rat (FO). Genotyping analysis of hCOL1A1 gene-edited rat was performed with primers SEQ ID NO: 40 to SEQ ID NO: 43, and genotyping analysis of hCOL1A2 gene-edited rat was performed with primers SEQ ID NO: 44 to SEQ ID NO: 47. The genotyping analysis described above is well-known technique in the art, and is not described here.

### Experiment 2: Gene-Edited Rat Breeding Method (Strategies I and II)

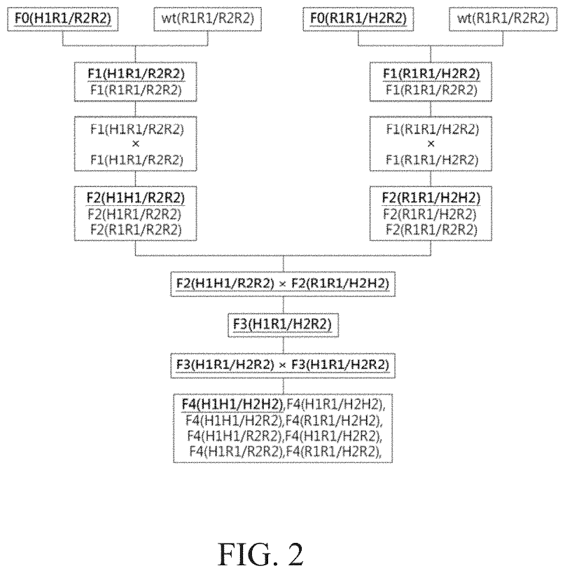

In order to obtain a homologous strain of gene-edited rat having human collagen, two breeding strategies were used to obtain the desired gene-edited animals. Please refer to FIG. 2, the breeding strategy I was described below. Taking hCOL1A1 gene-edited rat as an example, the F0 rat obtained in the experiment 1 had only one gene substitution on the genome (heterologous strain), and its genotype was represented by H1R1/R2R2 (H: human, R: rat, 1: COL1A1 gene, 2: COL1A2 gene). For obtaining a homologous strain, the following breeding procedure was performed: F0 (H1R1/R2R2) was mated with wild-type rat (R1R1/R2R2), i.e., F0 (H1R1/R2R2)×wt (R1R1/R2R2), to obtain the first generation, whose genotype may be F1 (H1R1/R2R2) or F1 (R1R1/R2R2). F1 (H1R1/R2R2) was self-mated, i.e., F1 (H1R1/R2R2)×F1 (H1R1/R2R2), to obtain progeny F2 (H1H1/R2R2), F2 (H1R1/R2R2) and F2 (R1R1/R2R2). The progeny F2 (H1H1/R2R2) was selected for subsequent breeding. The collagen gene COL1A2 gene-edited rat was mated in the same way as above, and the progeny F2 (R1R1/H2H2) was finally selected for subsequent breeding.

F2 (H1H1/R1R1) of COL1A1 gene-edited rat was mated with F2 (R1R1/H2H2) of COL1A2 gene-edited rat to obtain F3 (H1R1/H2R2). F3 (H1R1/H2R2) was self-mated and progeny F4 (H1H1/H2H2) was picked. A gene-edited rat in which COL1A1 and COL1A2 were both replaced would be obtained by self-mating of F4 (H1H1/H2H2), i.e., F4 (H1H1/H2H2)×F4 (H1H1/H2H2). The genotypes of the above gene-edited rat were obtained by genotyping using primers SEQ ID NO: 40 to SEQ ID NO: 43 (hCOL1A1 was delivered) and SEQ ID NO: 44 to SEQ ID NO: 47 (hCOL1A2 was delivered).

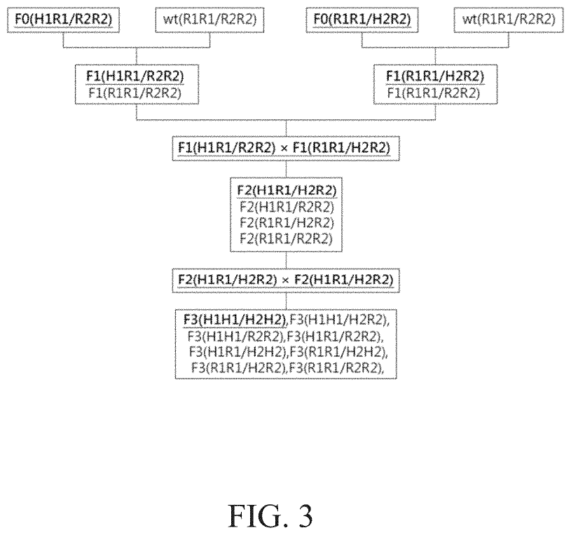

Referring to FIG. 3, the breeding strategy II for obtaining a homologous strain of gene-edited rat having human collagen was described below: F0 (H1R1/R2R2) rat obtained from experiment 1 was mated with wild-type rat (wild-type, R1R1/R2R2), i.e., F0 (H1R1/R2R2)×wt (R1R1/R2R2), to obtain first generation F1 (H1R1/R2R2) or F1 (R1R1/R2R2). The collagen gene COL1A2 gene-edited rat was mated in the same way as above, and F1 (R1R1/H2R2) or F1 (R1R1/R2R2) was obtained.

The first generation hCOL1A1 gene-edited rat F1 (H1R1/R2R2) was mated with the first generation hCOL1A2 gene-edited rat F1 (R1R1/H2R2), and the progeny whose genotype may be F2 (H1R1/H2R2), F2 (H1R1/R2R2), F2 (R1R1/H2R2), or F2 (R1R1/R2R2) was obtained. F2 (H1R1/H2R2) was self-mated, i.e., F2 (H1R1/H2R2)×F2 (H1R1/H2R2), and progeny F3 (H1H1/H2H2) was picked. By self-mating of F3 (H1H1/H2H2), i.e., F3 (H1H1/H2H2)×F3 (H1H1/H2H2), a gene-edited rat in which COL1A1 and COL1A2 were both replaced would be obtained. The genotypes of the above gene-edited rat were obtained by genotyping using primers SEQ ID NO: 40 to SEQ ID NO: 43 (hCOL1A1 was delivered) and SEQ ID NO: 44 to SEQ ID NO: 47 (hCOL1A2 was delivered).

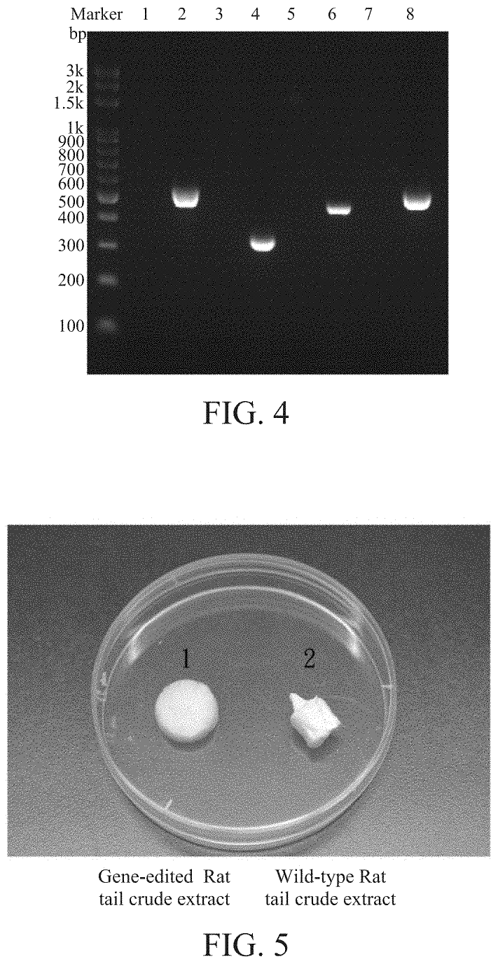

As shown in FIG. 4, for detecting whether the genomic DNA has the deoxyribonucleic acid sequences of SEQ ID NOs: 48 and 49 by the polymerase chain reaction, the genomic DNA of the gene-edited rat, in which COL1A1 and COL1A2 were both replaced, or that of wild-type rat, was used as a template, and SEQ ID NO: 52 and SEQ ID NO: 53, and SEQ ID NO: 55 and SEQ ID NO: 54 were used as primers. The gel electrophoresis results were shown in columns 1 to 4 of FIG. 4. The genomic DNA of the gene-edited rat has 520 bp of SEQ ID NO: 48 and 323 bp of SEQ ID NO: 49, confirming that the collagen gene COL1A1 in the gene-edited rat's genome was indeed a chimeric gene of the rat collagen gene COL1A1 and the human collagen gene COL1A1. For detecting whether the genomic DNA has the deoxyribonucleic acid sequences of SEQ ID NOs: 50 and 51 by the polymerase chain reaction, SEQ ID NO: 56 and SEQ ID NO: 57, and SEQ ID NO: 58 and SEQ ID NO: 59 were used as primers, and the genomic DNA of the gene-edited rat, in which COL1A1 and COL1A2 were both replaced, or that of wild-type rat, was used as a template. The gel electrophoresis results in columns 5 to 8 of FIG. 4 showed that the genomic DNA of the gene-edited rat has 447 bp of the SEQ ID NO: 50 and 521 bp of SEQ ID NO: 51, confirming that the collagen gene COL1A2 of the genome in the gene-edited rat's genome was indeed a chimeric gene of the rat collagen gene COL1A2 and the human collagen gene COL1A2.

Experiment 3: Preparation of Crude Extracts from Rat Tail

The crude extract of rat collagen can be extracted from the tail or skin of the rat. The following was the result of extraction and analysis by using the tail as material. After the rat was sacrificed, the tail was cut from the root and stored in −80° C. The tail of the gene-edited rat and the tail of the wild-type rat showed no significant difference in appearance (results not shown). The tail was thawed on ice for at least 30 minutes, and then the epithelial tissue was removed. The tail was roughly cut into six equal parts, and the bones and muscles were removed. The tendon was cut into fragment and soaked in water. After being washed with 1× phosphate-buffered saline buffer, it was soaked in 70% ethanol for 10 minutes. Subsequently, the non-tendon tissue (such as blood vessels or muscles) was removed, and the tendon tissue was sedimented by centrifugation. Further, 70% ethanol was removed, and the primary product of the tendon was obtained.

The primary product of the tendon was weighted, and 1 N acetic acid solution was added under stirring (the ratio of the weight of the primary product of the tendon to the volume of 1 N acetic acid solution was 1:99 to 1:9, preferably 1:9). The mixture was statically placed at 4° C. for 12 to 48 hours for reaction. After that, the solution of the primary product of the tendon was placed at −80° C. for 12 hours, and then lyophilized at −10° C. for 48 hours to remove acetic acid and water. Finally, a spongy freeze-dried crude extract was obtained as shown in FIG. 5 (i.e., the crude extract of the present disclosure), and this crude extract will be subjected to further analysis.

### Experiment 4: Polyacrylamide Gel Electrophoresis (PAGE) and Western Blotting (Western's Blotting)

The crude extract was dissolved in a 0.1 M lactic acid solution containing 10% sucrose to be a solution having the crude extract in a concentration of 1 mg/mL. Further, 5 μg of the crude extract was taken and analyzed by well-known non-denaturing polyacrylamide gel electrophoresis and Western blotting, briefly described as follows.

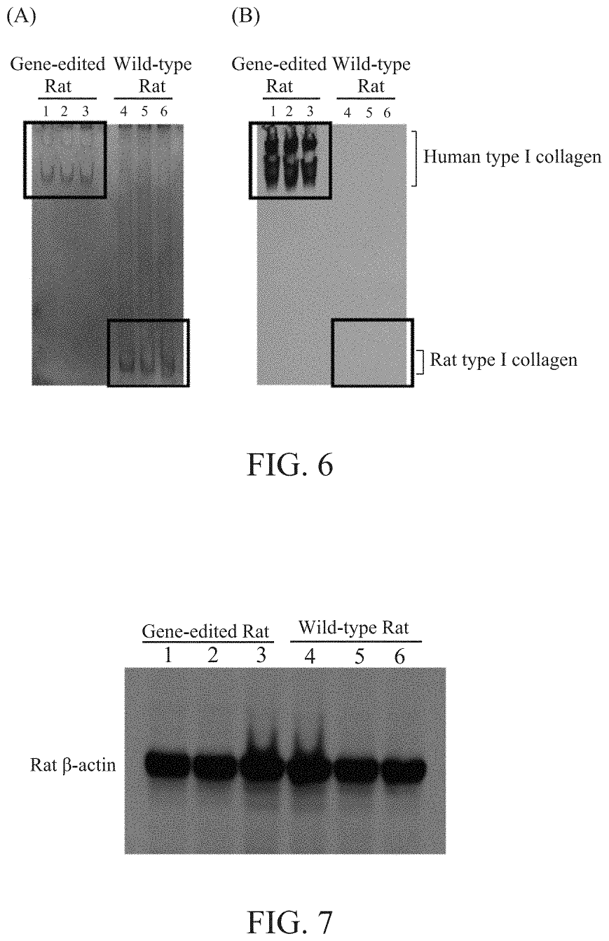

For non-denaturing polyacrylamide gel electrophoresis (Native PAGE), the proteins were dissolved in the gel, stained with a solution containing Coomassie blue dye for 2 hours, and then washed with a destaining solution for 24 hours. As shown in FIG. 6, the left (A) portion, the samples in columns 1 to 3 were the tail crude extract of gene-edited rat, and those in columns 4-6 were the tail crude extract of wild-type rat. After electrophoresis, two kinds of rat tail crude extract presented different patterns, and it thus can be preliminarily concluded that the tail crude extract of the gene-edited rat contained a large amount of certain protein, which was not present in the tail crude extract of wild-type rat.

To further confirm what the protein detected by non-denaturing gel electrophoresis was, the crude extract was analyzed by Western blotting below: after non-denaturing polyacrylamide gel electrophoresis (Native PAGE), the protein was transferred to the NC membrane (nitrocellulose membrane), and recognized by an antibody that specifically recognizes human type I collagen (Millipore antibody, number MAB3391). As shown in FIG. 6, the right (B) portion, columns 1 to 3 referred to the tail crude extract of gene-edited rat, wherein there was signal generated by the interaction with human type I collagen antibody, and the signal pattern could correspond to that shown in FIG. 6, the left (A) portion, confirming that the tail crude extract of the gene-edited rat contained a large amount of human type I collagen. On the contrary, in columns 4 to 6 of FIG. 6, the right (B) portion, there was no signal generated by the interaction with human type I collagen antibody, confirming that the tail crude extract of wild-type rat did not contain human type I collagen. In addition, it could be confirmed from the results of FIG. 6, the left (A) portion that there was no rat type I collagen in the gene-edited rat, indicating that the COL1A1 and COL1A2 of the gene-edited-rat have been replaced with hCOL1A1 and hCOL1A2, and the rat type I collagen was not expressed.

In addition, the crude extract of the rat tail was dissolved by sodium dodecyl sulfate polyacrylamide gel electrophoresis (SDS PAGE), and then the expression of the rat β-actin was detected by Western blotting. As shown in FIG. 7, both of the crude extracts of the gene-edited rat and the wild-type rat contained rat β-actin.

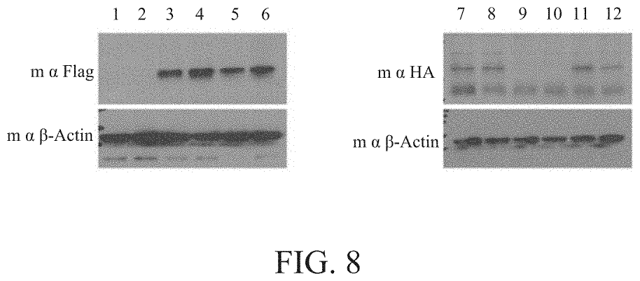

In addition, the crude extract of the rat tail was dissolved by sodium dodecyl sulfate polyacrylamide gel electrophoresis (SDS PAGE), and then Western blotting using anti-α Flag and anti-α HA monoclonal antibodies was performed to detect the amino acid sequence of SEQ ID NO: 60 synthesized by the 3′ ribonucleic acid sequence of the rat collagen gene COL1A1 and the Flag Tag ribonucleic acid sequence, and the amino acid sequence of SEQ ID NO: 61 synthesized by the 3′ ribonucleic acid sequence of COL1A2 and HA Tag ribonucleic acid sequence in the gene-edited rat, respectively. As shown in columns 3 to 5 of FIG. 8, the 29 kDa non-native chimeric protein peptide chain of SEQ ID NO: 60 was indeed present in the crude extract of gene-edited rat; and as shown in columns 7, 8, 11 and 12 of FIG. 8, the 28 kDa non-native chimeric protein peptide chain of SEQ ID NO: 61 was indeed present in the crude extract of the gene-edited rat; however, neither the non-native chimeric protein peptide chain SEQ ID NO: 60 nor SEQ ID NO: 61 was detected in the wild-type rat. As a result, it was confirmed that the gene-edited rat certainly expressed the chimeric protein peptide chain of the 3′ end sequence of the rat collagen gene COL1A1 and the Flag Tag sequence, and the chimeric protein peptide chain of the 3′ end sequence of the rat collagen gene COL1A2 and the HA Tag sequence.

The gene-edited rat obtained in the present disclosure expresses human type I collagen, but does not express rat type I collagen, and thus it can be avoided that rat type I procollagen peptide chain and human type I procollagen peptide chains are entangled into three helices, which will cause death of rats.

The crude extract obtained in the present disclosure can be further treated with pepsin or dispase to purify human collagen for further application in products using collagen as a raw material.

In an embodiment of the present disclosure, human collagen purified from the crude extract may be applied in a medical material such as collagen hemostatic cotton, absorbable surgical suture, wound dressing, bone filler, artificial blood vessel, soft tissue filler, and the like.

In an embodiment of the present disclosure, the human collagen purified from the crude extract may be applied in a cosmetic care product in the form of, but not limited to, a liquid, an emulsion, a cream, a powder, a whitening agent, a spotting agent, and a freckle agent or any combination of the above, such as a collagen moisturizer, a collagen moisturizer, a collagen mask, and the like.

In an embodiment of the present disclosure, when the human collagen purified from the crude extract is applied in a food additive, it may be added to, for example, a nutraceutical such as bone joint health care and anti-aging health care.

Based on the above examples and description, the present disclosure has the following advantages over the prior art:

1. The crude extract of the present disclosure can be further purified to obtain human type I collagen, which can be used for medical or cosmetic purposes to reduce the incidence of allergy to non-human collagen.

2. The crude extract of the present disclosure is derived from a gene-edited rat whose post-translational modification is more similar to the native state of human type I collagen.

3. The crude extract of the present disclosure is derived from experimental animals, whose culture environment is closely monitored, such that the concern of infectious substances in raw materials can be reduced, so as to provide a source of high quality collagen.

In summary, a protein crude extract of the present disclosure isolated from non-human connective tissue, and methods and uses thereof, can indeed achieve the desired effect by the above disclosed embodiments.

A person having ordinary skill in the art will appreciate that the above-described examples are merely exemplary embodiments, and various changes, substitutions and alterations may be made without departing from the spirit and scope of the present disclosure.

Incorporated by reference is information in accompanying computer readable formatted document saved as “SEQUENCELISTING370.txt” and entitled “80036US -Sequence listing-hana-ycl-20191211 SEQUENCE LISTING” and containing 61 sequences, created on Dec. 11, 2019 and having a size of 57 kilobytes. The computer readable formatted document saved as “SEQUENCELISTING370.txt” and entitled “80036US-Sequence listing-hana-ycl-20191211 SEQUENCE LISTING” is part of this disclosure.

1. A protein crude extract isolated from a connective tissue of a non-human mammal, wherein the non-human mammal has a genomic DNA comprising a deoxyribonucleic acid sequence encoding a chimeric collagen peptide chain and does not have wild-type collagen of the non-human mammal, and wherein the protein crude extract has human type I collagen and a non-native chimeric protein peptide chain.

2. The protein crude extract of claim 1, further comprising β-actin of the non-human mammal.

3. The protein crude extract of claim 1, wherein the chimeric collagen peptide chain comprises at least one portion of an amino acid sequence of the human type I collagen, and at least one portion of an amino acid sequence of N-terminal and/or C-terminal peptide of the wild-type collagen of the non-human mammal.

4. The protein crude extract of claim 1, wherein the non-native chimeric protein peptide chain has an amino acid sequence comprising SEQ ID NO: 60 or SEQ ID NO: 61.

5. The protein crude extract of claim 3, wherein the deoxyribonucleic acid sequence comprises at least one deoxyribonucleic acid sequence selected from the group consisting of SEQ ID NO: 48, SEQ ID NO: 49, SEQ ID NO: 50, SEQ ID NO: 51, SEQ ID NO: 6 and SEQ ID NO: 7.

6. (canceled)

7. The protein crude extract of claim 1, which comprises:
(i) an amino acid sequence having a similarity of at least 91% as compared with SEQ ID NO: 1 and an amino acid sequence of SEQ ID NO: 60;
(ii) an amino acid sequence having a similarity of at least 93% as compared with SEQ ID NO: 2 and an amino acid sequence of SEQ ID NO: 61; or
(iii) an amino acid sequence having a similarity of at least 91% as compared with SEQ ID NO: 1, an amino acid sequence having a similarity of at least 93% as compared with SEQ ID NO: 2, and an amino acid sequence of SEQ ID NO: 60 or SEQ ID NO: 61.

8. (canceled)

9. The protein crude extract of claim 1, which is specifically recognized by an anti-human collagen antibody.

10. A method for preparing a protein crude extract containing a target amino acid sequence, comprising:
(a) constructing a DNA sequence construct encoding human type I collagen;
(b) microinjecting the DNA sequence construct into an embryo of a non-human mammal, and transplanting the embryo of the non-human mammal into a female animal of a same species to develop the embryo of the non-human mammal into a mature animal, wherein the mature animal has a genomic DNA comprising a deoxyribonucleic acid sequence encoding a chimeric collagen peptide chain and does not have wild-type collagen of the non-human mammal; and
(c) isolating the protein crude extract containing the target amino acid sequence from a connective tissue of the mature animal, wherein the target amino acid sequence comprises human type I collagen.

11. The method of claim 10, wherein the target amino acid sequence further comprises β-actin of the mature animal.

12. (canceled)

13. The method of claim 10, wherein the target amino acid sequence comprises:
(i) an amino acid sequence having a similarity of at least 91% as compared with SEQ ID NO: 1 and an amino acid sequence of SEQ ID NO: 60;
(ii) an amino acid sequence having a similarity of at least 93% as compared with SEQ ID NO: 2 and an amino acid sequence of SEQ ID NO: 61; or
(iii) an amino acid sequence having a similarity of at least 91% as compared with SEQ ID NO: 1, an amino acid sequence having a similarity of at least 93% as compared with SEQ ID NO: 2, and an amino acid sequence of SEQ ID NO: 60 or SEQ ID NO: 61.

14. (canceled)

15. The method of claim 10, wherein the chimeric collagen peptide chain comprises at least one portion of an amino acid sequence of the human type I collagen, and at least one portion of an amino acid sequence of N-terminal and/or C-terminal peptide of the wild-type collagen of the non-human mammal.

16. The method of claim 15, wherein the deoxyribonucleic acid sequence encoding the chimeric collagen peptide chain comprises at least one deoxyribonucleic acid sequence selected from the group consisting of SEQ ID NO: 48, SEQ ID NO: 49, SEQ ID NO: 50, SEQ ID NO: 51, SEQ ID NO: 6 and SEQ ID NO: 7.

17. The method of claim 10, wherein the non-human mammal is a rat.

18. The method of claim 10, wherein the protein crude extract is specifically recognized by an anti-human collagen antibody.

19.-31. (canceled)

32. A non-human gene-edited mammal having a genomic DNA comprising a deoxyribonucleic acid sequence encoding a chimeric collagen peptide chain, wherein the non-human gene-edited mammal does not have wild-type collagen of the non-human gene-edited mammal.

33. The non-human gene-edited mammal of claim 32, wherein the chimeric collagen peptide chain comprises at least one portion of an amino acid sequence of human type I collagen, and at least one portion of an amino acid sequence of N-terminal and/or C-terminal peptide of the wild-type collagen of the non-human gene-edited mammal.

34. The non-human gene-edited mammal of claim 32, wherein the deoxyribonucleic acid sequence comprises at least one deoxyribonucleic acid sequence selected from the group consisting of SEQ ID NO: 48, SEQ ID NO: 49, SEQ ID NO: 50, SEQ ID NO: 51, SEQ ID NO: 6 and SEQ ID NO: 7.

35. The non-human gene-edited mammal of claim 32, comprising:
(i) an amino acid sequence having a similarity of at least 91% as compared with SEQ ID NO: 1 and an amino acid sequence of SEQ ID NO: 60;
(ii) an amino acid sequence having a similarity of at least 93% as compared with SEQ ID NO: 2 and an amino acid sequence of SEQ ID NO: 61; or
(iii) an amino acid sequence having a similarity of at least 91% as compared with SEQ ID NO: 1, an amino acid sequence having a similarity of at least 93% as compared with SEQ ID NO: 2, and an amino acid sequence of SEQ ID NO: 60 or SEQ ID NO: 61.

36. (canceled)

37. The non-human gene-edited mammal of claim 32, which is a rat, a mouse, a pig, a rabbit, a sheep, a goat, a cat, a dog, a calf or a baboon.

38. The non-human gene-edited mammal of claim 37, which is a rat.
